(19) United States
(12) Patent Application Publication
(10) Pub. No.: US 2023/0355644 A1
(43) Pub. Date: Nov. 9, 2023

(54) ACTIVE INGREDIENTS AND DRUGS FOR THE THERAPY OF VIRAL DISEASES, ESPECIALLY CORONA INFECTIONS, ESPECIALLY COVID-19

(71) Applicant: Creative Therapeutics GmbH, Wuppertal (DE)

(72) Inventor: Hans-Joachim ZEILER, Wuppertal (DE)

(21) Appl. No.: 18/246,383

(22) PCT Filed: Sep. 6, 2021
(86) PCT No.: PCT/EP21/74443

§ 371 Date: Mar. 23, 2023

(30) Foreign Application Priority Data
Sep. 25, 2020 (DE) 10 2020 125 125.4
Oct. 1, 2020 (DE) 10 2020 125 641.8

(51) Int. Cl. A61K 31/655; A61P 31/14; A61K 45/06

(52) U.S. Cl. CPC A61K 31/655; A61P 31/14; A61K 45/06

(57) ABSTRACT

The present invention relates to the field of therapy of viral infections (virus infections), especially infections with corona viruses, especially COVID-19; especially, the present invention relates to active ingredients and medicaments for use in such therapy.

### CROSS-REFERENCES TO RELATED APPLICATIONS

This application is a National Stage filing of International Application Application PCT/EP 2021/074443(WO 2022/063549) filed Sep. 6, 2021, entitled “ACTIVE SUBSTANCES AND DRUGS FOR THERAPY OF VIRAL DISEASES, PARTICULARLY CORONA INFECTIONS, MORE PARTICULARLY COVID-19” claiming priority to DE 10 2020 125 125.4 filed Sep. 25, 2020, and DE 10 2020 125 641.8 filed Oct. 1, 2020. The subject application claims priority to DE 10 2020 125 125.4, DE 10 2020 125 641.8, and PCT/EP 2021/074443 and incorporates all by reference herein, in their entirety.

### BACKGROUND OF THE INVENTION

The present invention relates to the field of therapy of viral infections (virus infections), especially infections with corona viruses, especially COVID-19. Especially, the present invention relates to active ingredients and drugs for use in such therapy.

Especially, the present invention relates to active ingredients and drugs for use in the prophylactic and/or therapeutic treatment of viral diseases, especially corona infections, especially COVID-19, or to the use of active ingredients and medicaments for the prophylactic and/or therapeutic treatment of viral diseases, especially corona infections, especially COVID-19. In the context of the present invention, special active ingredients based on diphenyl derivatives or medicaments containing these special active ingredients are used, as defined in the following description and in the patent claims relating to the present invention.

Coronaviridae is a family of viruses within the order Nidovirales. The viruses within this virus family are also known colloquially as coronaviruses and are among the RNA viruses with the largest genomes. The first coronaviruses were discovered and described as early as the mid-1960s. The roughly spherical viruses in the electron microscope image are conspicuous by a ring of petal-like projections reminiscent of a solar corona, which gave this virus family its name.

Representatives of the Coronaviridae family of viruses cause very different diseases in all four classes of terrestrial vertebrates (i.e. mammals, birds, reptiles, and amphibians). They are highly genetically variable and thus can infect multiple species of hosts. In humans, seven species of coronaviruses are important as pathogens of mild respiratory infections (especially colds or flu-like infections) to the so-called severe acute respiratory syndrome (SARS or Severe Acute Respiratory Syndrome).

Among human coronaviruses, the following have become particularly well known: SARS-CoV-1 (Severe Acute Respiratory Syndrome Coronavirus-1), MERS-CoV (Middle East Respiratory Syndrome Coronavirus), and SARS-CoV-2 (Severe Acute Respiratory Syndrome Coronavirus 2 or COVID-19). These coronaviruses are the triggers of the 2002/2003 SARS pandemic, the 2012 MERS epidemic, and the 2019 COVID-19 pandemic, respectively.

COVID-19 (i.e. the abbreviation for English Coronavirus-Disease 2019 or German Coronavirus-Krankheit-2019, colloquially also referred to as coronavirus disease or the like) is an infectious disease resulting in infection with the novel coronavirus SARS-CoV-2. Primarily affecting the respiratory tract, the disease was first described in Wuhan, China, in late 2019, then developed into an epidemic first in the People's Republic of China in January 2020, and eventually spread globally to become the COVID-19 pandemic.

Infection with COVID-19 usually occurs through droplet transmission. The incubation period of COVID-19 is on average five to six days, although up to two weeks can pass between infection and the appearance of the first symptoms, and in isolated cases the first symptoms can appear within 24 hours of infection with SARS-CoV-2. The most common symptoms are fever, dry cough, and fatigue; less common symptoms include muscle pain, nasal congestion, headache, conjunctivitis, sore throat, diarrhea, loss of taste or smell, or skin rash or discoloration of fingers or toes. Infected individuals without symptoms may still be potential carriers of coronavirus. If the course of the disease is mild, symptoms generally resolve within two weeks. If COVID-19 is more severe, convalescence may last three to six weeks or even longer. In about 81% of registered infections, the course of the disease is mild, with fever or mild pneumonia; however, in about 14% of cases, the course is more severe, and in about 5% of cases, the course is even so severe that patients require intensive care.

COVID-19 is currently the subject matter of intensive research. Efficient and specific causal therapies or vaccines are currently not available.

Like SARS-CoV-1 in SARS, the COVID-19-causing virus SARS-CoV-2 typically enters the human cell via binding to the cell membrane-anchored enzyme ACE2 (angiotensin-converting enzyme 2), whereby the viral spike protein interacts with ACE2. This process requires the involvement of the serine protease TMPRSS2 (transmembrane serine protease 2). In experiments with HeLa cells expressing ACE2 from humans, chinese horseshoe bats (Rhinolophus sinicus), a creeping cat species, domestic pigs and mice, SARS-CoV-2 was able to use the respective ACE2 protein as a receptor to enter the cell; only the mouse ACE2 enzyme failed to do so—as did HeLa cells, which do not produce ACE2. In contrast, SARS-CoV-2 does not bind to receptors used by other coronaviruses.

In addition to the pathway via ACE2, an alternative penetration pathway has been experimentally demonstrated in T-lymphocytes that carry little or no ACE2 on their surface: The virus penetrates these cells via a spike protein-mediated fusion of the viral membrane with the lymphocyte cell membrane.

A reverse search in a human gene database (Human Cell Atlas or HCA for short) for cell type and tissues in which TMPRSS₂ is also present on membrane surfaces in addition to ACE2 shows that the highest concentrations of these two proteins occur in the nasal mucosa, especially in the goblet cells, but also in the ciliated epithelia, which is why these cells are regarded as a portal of entry for SARS-CoV-2 and are also suspected to be a reservoir. The proteins are also produced in the corneal cells of the eye, in the intestinal mucosa and in the heart in pericytes of the blood capillaries, cardiac muscle cells and fibroblasts. The first phase of the attack in the nasopharynx remains almost symptom-free, whereas the lungs are predominantly attacked in the transition to a severe form of progression, since a large proportion of the ACE-2-expressing cells in humans are found in the type II pneumocytes of the lungs. Other reasons given for the particular susceptibility of the lung are its large surface area; in addition, ACE-2-expressing pneumocyte type II cells possess diverse genes that favor replication and transmission of SARS-CoV-2.

In studies of cryopreserved lung tissue samples from non-infected individuals, it can also be shown that lung tissue hardly produces ACE2 as well as the transmembrane protease TMPRSS2, whereas pneumocytes type II in the lung are increased. These progenitor cells tend to be increased in men and in advanced age. In addition to different ACE2 levels in men and women, a cause for the different severity of the disease is suspected in the gender-specific hormone balance: estrogen promotes an immune response, whereas testosterone suppresses it.

Recent evidence also shows that the proprotease furin is co-expressed in the lung epithelium and adjacent tissue cells, which in turn facilitates cell entry for the virus by providing a furin-specific separation site at the spike protein.

In addition to the lungs, ACE2 has also been detected in the small and large intestines, the respiratory tract and the kidneys. Multiplication of the virus in intestinal cells was also confirmed.

In terms of clinical symptoms and laboratory signs of illness, it is difficult to distinguish it from other viral diseases, such as influenza, based on symptoms alone. After an incubation period of typically five to six days, in rare cases up to 14 days, fever, muscle pain and a dry cough may occur—as described at the beginning. Frequently, the disease also manifests itself with a general feeling of severe illness. As the disease progresses, severe shortness of breath may then develop due to infection of the lower respiratory tract, culminating in pneumonia. This may be accompanied by chest pain in the form of pleurisy. Approximately 85% of severely ill COVID-19 patients develop lymphopenia. The severely ill patients also frequently develop hypercytokinemia (so-called cytokine storm), whereby this cytokine storm results from an overreaction of the immune system; this overreaction is characterized by a marked increase in inflammation-relevant cytokines, such as especially interleukin-6, interleukin-8, interleukin-1s, and TNF-α. The increased release of these cytokines leads to an overproduction of immune cells, especially in the lung tissue.

Especially, the diagnosis of COVID-19 can be made by laboratory diagnostic detection, especially by means of specific viral and antibody detections.

An efficient and specific causal therapy has not yet been established. The nucleoside analog Remdesivir shows a reduction of disease duration in hospitalized patients in a preliminary published randomized study. This medicament is approved in the European Union for COVID-19 patients requiring oxygen and can be considered for critically ill patients according to the DIVI guideline (Deutsche Interdisziplinäre Vereinigung für Intensiv-und Notfallmedizin e.V.).

Chloroquine and hydroxychloroquine, on the other hand, show no evidence of efficacy—contrary to original expectations.

Tocilizumab, a monoclonal antibody approved for the treatment of various forms of rheumatoid arthritis and cytokine release syndrome, among others, has also been shown to be ineffective.

Antibody-rich plasma from recovered patients, on the other hand, appears to be suitable for treating acute cases, but can demonstrate success only in the early phase of the disease.

Further studies show that dexamethasone reduces the mortality rate from 41% to 29% in patients on ventilators and from 26% to 23% in patients on oxygen. In patients who do not require oxygen administration, however, treatment with dexamethasone shows no positive effect. Dexamethasone is supposed to reduce an excessive reaction of the immune system, especially the so-called cytokine storm.

In severely ill COVID-19 patients, administration of low molecular weight heparin is also recommended to reduce the risk of thrombosis and pulmonary embolism. Due to the high incidence of pulmonary embolism and leg vein thrombosis associated with severe courses of COVID-19, the administration of more potent anticoagulants is being considered; however, sufficient data on benefit and risk are not yet available.

To date, it is also still unclear to what extent a passed-through infection confers immunity; so far, based on findings from animal models, it is assumed that acute immunity exists, although it is not certain how long this lasts. The state of research on late effects and estimation of mortality risk is also still uncertain.

As a result, most measures worldwide are aimed at the prevention of COVID-19 diseases, especially through increased individual hygiene measures, such as the wearing of mouth/nose protection, hand disinfection, surface cleaning etc., since an established causal therapy for curative treatment has so far been established or vaccination for prevention purposes does not yet exist.

### BRIEF SUMMARY OF THE INVENTION

Against this background, one object of the present invention is to provide an efficient, preferably causal, therapy for viral diseases or viral infections (virus infections), especially infections with corona viruses, especially COVID-19.

Especially, one object of the present invention is to find or provide active ingredients and medicaments containing these active ingredients which are suitable for use in the prophylactic and/or therapeutic treatment of viral diseases or viral infections (viral infections), especially corona infections, especially COVID-19, preferably as part of an efficient, especially causal, therapy.

In a completely surprising way, however, the applicant has now discovered that special active ingredients based on diphenyl derivatives, as defined in more detail in the following description and in the patent claims of the present invention, and which have so far only been considered or used in the therapy of tumor or cancer diseases, are unexpectedly also suitable in an efficient way for the preferred causal therapy of viral diseases or viral infections (viral infections), especially infections with corona viruses, especially COVID-19.

To solve the problem described above, the present invention therefore proposes—according to a first aspect of the present invention—active ingredients or diphenyl derivatives for use in the prophylactic and/or therapeutic treatment of viral infections (virus infections), especially infections with corona viruses, especially COVID-19, according to patent claim 1. Advantageous further developments and embodiments of this aspect of the invention are provided below.

A further subject matter of the present invention—according to a second aspect of the present invention—is the use according to the invention of the aforementioned active ingredients or diphenyl derivatives for the prophylactic and/or therapeutic treatment of viral infections (virus infections), especially infections with corona viruses, especially COVID-19, or the use according to the invention of the aforementioned active ingredients or diphenyl derivatives for the preparation of a drugs or medicament for the prophylactic and/or therapeutic treatment of viral infections (virus infections), especially infections with corona viruses, especially COVID-19.

Equally the subject matter of the present invention—according to a third aspect of the present invention—is the use according to the invention of the aforementioned active ingredients or diphenyl derivatives as antiviral agents for the prophylactic and/or therapeutic treatment of viral diseases, especially corona infections, especially COVID-19, respectively for the preparation of an antiviral agent for the prophylactic and/or therapeutic treatment of viral diseases, especially corona infections, especially COVID-19. Advantageous further developments and embodiments of this aspect of the invention are also provided.

Yet another subject matter of the present invention—according to a fourth aspect of the present invention—is a method for the prophylactic and/or therapeutic treatment of viral diseases, especially corona infections, especially COVID-19. Advantageous further developments and embodiments of this aspect of the invention are further discussed.

Also the subject matter of the present invention—according to a fifth aspect of the present invention—is a drug or medicament for (use in the) prophylactic and/or therapeutic treatment of viral diseases, especially corona infections, especially COVID-19.

Finally, the subject matter of the present invention—according to a sixth aspect of the present invention—is equally a pharmaceutical combination for (use in the) prophylactic and/or therapeutic treatment of viral diseases, especially corona infections, especially COVID-19.

It goes without saying that features, embodiments, advantages and the like, which are subsequently listed below only with regard to one aspect of the invention for the purpose of avoiding repetition, naturally also apply accordingly with respect to the other aspects of the invention, without this requiring separate mention.

Furthermore, with regard to the following description of the present invention, it is also the case that the features of the present invention specified in connection with the specific features, embodiments, advantages, examples or the like are also deemed to be disclosed in their combination. Thus, in the present case, superordinate combinations of individual or several features, which are specified for respective features, embodiments, examples or the like, are also considered disclosed herein.

### BRIEF DESCRIPTION OF THE DRAWINGS

FIG. 1A illustrates the differentially regulated gene expression of genes involved in cell cycle regulation, drug membrane transport, drug metabolism, cell adhesion and other selected metabolic pathways in UWB1,289 cells after treatment with compound (IV-A3).

FIG. 1B illustrates the differential gene expression of genes involved in metabolic pathways regulating DNA repair in UWB1,289 cells after treatment with compound (IV-A3).

FIG. 2A provides the mean values from each of three independent experiments with SARS-CoV-2 infected Caco-2 cells demonstrating a significant decrease in viral load in treated cells compared to untreated cells with all compounds.

FIG. 2B provides the mean values from each of three independent experiments with SARS-CoV-2 infected Caco-2 cells demonstrating a significant decrease in viral load in treated cells compared to untreated cells with all compounds.

FIG. 3A provides the mean values from each of three independent experiments with SARS-CoV-2 infected Vero-E6 cells demonstrating a significant decrease in viral load in treated cells compared to untreated cells with all compounds.

FIG. 3B provides the mean values from each of three independent experiments with SARS-CoV-2 infected Vero-E6 cells demonstrating a significant decrease in viral load in treated cells compared to untreated cells with all compounds.

### DETAILED DESCRIPTION OF THE INVENTION

Thus, a subject matter of the present invention—according to a first aspect of the present invention—is a diphenyl derivative for use in the prophylactic and/or therapeutic treatment of viral diseases, especially corona infections, especially COVID-19,

wherein the diphenyl derivative contains per molecule
(i) at least one dialkyltriazenyl group and
(ii) at least one sulfooxy group and/or at least one sulfamoyloxy group.

In the context of the present invention, the diphenyl derivative can be used as such or in the form of its salts, hydrates, solvates, free acids or solvates of these salts or solvates of these free acids or isomers, especially constitutional isomers, tautomers and stereoisomers, esters, prodrugs or metabolites and/or are present as physiologically tolerable salt(s), hydrate(s), solvate(s), free acid(s) or solvate(s) of these free acid(s) or isomer(s), especially constitutional isomer(s), tautomer(s) and stereoisomer(s), ester(s), prodrug(s) or metabolite(s).

The active ingredients or compounds (i.e. diphenyl derivatives) used in accordance with the invention as such, as well as their preparation or provision, are known in principle from the prior art (cf. especially documents WO 2012/122969 A1, DE 10 2015 008 631 A1, EP 2 557 075 A1 and WO 2009/004060 A1, wherein the entire disclosure content of these documents is hereby expressly incorporated by reference in their entirety).

However, the active ingredients or compounds described therein (i.e. diphenyl derivatives of the aforementioned type) have so far been considered or used exclusively for cancer and tumor therapy.

As already described at the beginning, the applicant has now for the first time—completely surprisingly and unexpectedly—found that these special active ingredients based on diphenyl derivatives are also suitable in an efficient manner for the preferably causal therapy of viral diseases or viral infections (virus infections), especially infections with corona viruses, especially COVID-19.

Many viruses use and manipulate certain signaling and communication pathways of their host cells to promote their own replication. In a completely surprising and unexpected way, the applicant has now discovered that the diphenyl derivatives in question can be used to disrupt or interrupt the relevant viral signaling or communication pathways, and that such disruption of these pathways means that the viruses can no longer reproduce.

One advantage of the fact that the applicant can draw on active ingredients which are known per se, but which have not yet been considered at all for the purpose of use or therapy according to the invention, is that these active ingredients are readily accessible and, above all, have a decisive development advantage over novel active ingredients, since numerous properties of the active ingredients have already been investigated, especially stability behavior, toxicities, pharmaceutical-pharmacological formulability and modes of application, etc. Therefore, any clinical studies for the purposes of antiviral therapy according to the invention can in principle begin immediately and without further extensive preliminary investigations.

As the applicant has discovered, the diphenyl derivatives used in accordance with the invention lead especially (without, however, wishing to commit to a particular theory) to the downregulation of certain repair systems in the cells. These systems are also used by viruses to generate a so-called DNA damage response (DDR), which drives the host cell into cell cycle arrest and apoptosis, with the decay products released in the process being used by the viruses to drive their own replication.

By down-regulating these cell repair systems, the viruses are deprived of the opportunity to multiply, so that on this basis, using the diphenyl derivatives employed according to the invention, an efficient, especially causal, therapy of viral diseases or viral infections (virus infections), especially infections with corona viruses, especially COVID-19, can be provided for the first time.

In other words, the applicant surprisingly found that the previously described shutdown of cell repair (mechanisms) not only damages tumor cells, but also blocks viral replication in infected host cells when viruses are deprived of the ability to access these systems.

Specific investigations regarding the shutdown of cell repair (mechanisms) have been performed by the applicant especially on ovarian cancer cells or ovarian cancer cell lines, but also on other cell systems and relevant models.

Due to the previously demonstrated mechanism of action, the diphenyl derivatives used according to the invention provide for the first time an efficient, especially causal, broad-spectrum antiviral therapy, or rather the diphenyl derivatives used according to the invention are unexpectedly suitable as a broad-spectrum antiviral agent (i.e. with regard to the therapy of infections caused by a large number of viruses, but especially also infections with corona viruses, especially COVID-19, since the latter especially rely on the previously described repair (mechanisms) of the host cells).

The diphenyl derivatives used according to the invention are distinguished from other antiviral agents (e.g. Remdesivir etc.) and especially also from other diphenyl derivatives by a better therapeutic range, but especially also by lower side effects with a high antiviral effect, especially also by improved uptake in cells which are preferentially attacked by coronaviruses or viruses of the SARS type (e.g. lungs, kidneys, heart, CNS etc.).

Among the diphenyl derivatives used according to the invention, those with a triazene group are particularly preferred. However, such triazene derivatives have so far only become known as agents with antitumor activity (e.g. dacarbazine or temozolomide), but have never come into wider use even as cytostatic agents (i.e. melanoma and glioblastoma, respectively); an indication outside of antitumor activity has so far remained unrecognized and therefore not considered.

The diphenyl derivatives used in accordance with the invention possess—in addition to their antitumor effect—completely surprisingly also a pronounced broad antiviral effect (e.g. DNA, RNA viruses), especially with regard to RNA viruses (e.g. SARS-based viruses). These compounds are particularly characterized by the fact that they have a very good tolerability and can be made accessible for therapy in humans and animals, especially for the therapy of severe viral diseases of the SARS type in the acute phase or for the prevention or containment of infection and the spread of the viruses in the body, for the prevention of life-threatening complications with long-term damage and death, etc.

Without wishing to commit to a particular theory, the novel medical indication or mode of action of the diphenyl derivatives used for the first time as antivirally active agents in the context of the present invention is based especially on the fact that the compounds used according to the invention in therapeutically effective doses or concentrations (e.g. 10-1,000 μmol/l) suppress the DNA expression of genes in the nucleus of the host cell which code for a number of important repair systems (HR; NER; . . . ), so that the invading virus, especially RNA virus, fails to generate the crucial metabolic shift at the DNA level in the nucleus of the host cell, namely to trigger a repair system-mediated DNA damage response (DDR) and consequently to force cell cycle arrest and apoptosis, which generally leads to the death of the host cell. The blockade or non-expression of important repair genes by the compounds used according to the invention thus deprives the virus of the basis for using these processes, which are essential for its own propagation.

Treatment of the host cells with the compounds of the invention thus leads to a shutdown of important repair systems, which the virus needs to initiate and maintain its own replication. Even the blockade of a single specific repair system has significant inhibitory effects on virus replication.

What is particularly surprising is that the compounds used according to the invention temporarily interfere broadly with the expression of a number of important repair systems, thus protecting the infected host cell from the transformation process of the host cell induced by the virus.

Consequently, the present invention and the therapy concept according to the invention are associated with a large number of advantages and special features.

With regard to the diphenyl derivative used according to the invention for the prophylactic and/or therapeutic treatment of viral diseases, especially corona infections, especially COVID-19, the diphenyl derivative contains—as described above—per molecule (i) at least one dialkyltriazenyl group and (ii) at least one sulfooxy group and/or at least one sulfamoyloxy group (see also patent claim 1).

As equally described above, in the context of the present invention, the diphenyl derivative can be used as such or in the form of its salts, hydrates, solvates, free acids or solvates of these salts or solvates of these free acids or isomers, especially constitutional isomers, tautomers and stereoisomers, esters, prodrugs or metabolites and/or are present as physiologically acceptable salt(s), hydrate(s), solvate(s), free acid(s) or solvate(s) of said salt(s) or solvate(s) of said free acid(s) or isomer(s), especially constitutional isomer(s), tautomer(s) and stereoisomer(s), ester(s), prodrug(s) or metabolite(s) (cf. patent claim 2).

As previously disclosed, it is thus a subject matter of the present invention—according to a first aspect of the present invention—to provide a diphenyl derivative for use in the prophylactic and/or therapeutic treatment of viral diseases, especially corona infections, especially COVID-19, wherein the diphenyl derivative contains per molecule
(i) at least one dialkyltriazenyl group and
(ii) at least one sulfooxy group and/or at least one sulfamoyloxy group.

In the context of the present invention, the diphenyl derivative can be used as such or can be present in the form of its salts, hydrates, solvates, free acids or solvates of these salts or solvates of these free acids or isomers, especially constitutional isomers, tautomers and stereoisomers, esters, prodrugs or metabolites and/or can be present as physiologically tolerable salt(s), hydrate(s), solvate(s), free acid(s) or solvate(s) of these free acid(s) or isomer(s), especially constitutional isomer(s), tautomer(s) and stereoisomer(s), ester(s), prodrug(s) or metabolite(s).

According to a particular embodiment of the present invention according to the present first aspect of the invention, the subject matter of the present invention is especially a diphenyl derivative for use in the prophylactic and/or therapeutic treatment of viral diseases, especially corona infections, especially COVID-19, especially a diphenyl derivative as previously defined (i.e. a diphenyl derivative according to claim 1 or claim 2),

wherein the diphenyl derivative corresponds to the general formula (1)

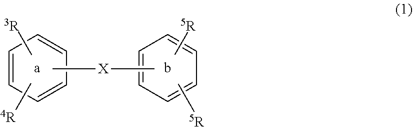

wherein:
X denotes a direct C—C bond between the rings a and b, CH₂, CHOH, CO, S, SO, SO₂, —N═N—, —CR⁷═CR⁸— or a divalent —C(O)—C*═CH—O— radical which, together with the two adjacent C-atoms of the ring a on which it is located, forms a pyranone ring, the ring b being located on the C* atom of this radical,
R³, R⁶ each independently of one another denote hydrogen, hydroxyl-, C₁-C₄-alkyl, C₁-C₄-alkoxy, C₁-C₄-alkyl-S—, C₁-C₄-alkyl-SO—, C₁-C₄-alkyl-SO₂—, halogen, nitro, cyano or a —OSO₂Y-group,
R⁴, R⁵ each independently of one another denote a —N═N—N(R²)₂-group or a —OSO₂Y-group,
R⁷ denotes hydrogen, methyl, ethyl or a phenyl radical which is substituted by the radicals R⁹, R¹⁰ and is linked directly to the C-atom bearing R⁷,
R⁸ denotes hydrogen, ethyl, CN, NO₂, —CH₂CH₂-halogen or CH₂CH₂OH,
R⁹, R¹⁰ each independently of one another denote hydrogen, hydroxyl, C₁-C₄-alkyl, C₁-C₄-alkoxy, halogen, nitro, cyano, a —N═N—N(R²)₂-group or a —OSO₂Y-group,
Y denotes OH or N(R¹)₂,
R¹ denotes hydrogen, methyl or ethyl,
R² denotes methyl or ethyl;
especially, however, with the proviso that per total molecule (1) there are one to two —N═N—N(R²)₂— and one to two —OSO₂Y-groups on any ring carbon atoms of aromatic rings;
as well as its salts, hydrates, solvates and free acids and solvates of these salts and solvates of these free acids and its isomers, especially constitutional isomers, tautomers and stereoisomers, esters, prodrugs and metabolites.

According to a further particular embodiment of the present invention in accordance with the present first aspect of the invention, it is especially also a subject matter of the present invention to provide a diphenyl derivative for use in the prophylactic and/or therapeutic treatment of virus diseases, especially corona infections, especially COVID-19, especially a diphenyl derivative as previously defined,

wherein the diphenyl derivative corresponds to the general formula (2)

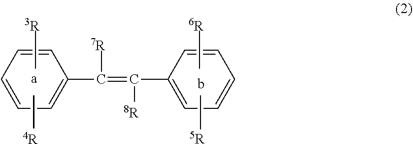

wherein:
R³, R⁶ each independently of one another denote hydrogen, hydroxyl, C₁-C₄-alkyl, C₁-C₄-alkoxy, C₁-C₄-alkyl-S—, C₁-C₄-alkyl-SO—, C₁-C₄-alkyl-SO₂—, halogen, nitro, cyano or a —OSO₂Y-group,
R⁴, R⁵ each independently of one another denote a —N═N—N(R²)₂ group or a —OSO₂Y group,
R⁷ denotes hydrogen, methyl, ethyl or a phenyl radical which is substituted by the radicals R⁹, R¹⁰ and is linked directly to the C-atom bearing R⁷,
R⁸ denotes hydrogen, ethyl, CN, NO₂, —CH₂CH₂-halogen or CH₂CH₂OH,
R⁹, R¹⁰ each independently of one another denote hydrogen, hydroxyl-, C₁-C₄-alkyl, C₁-C₄-alkoxy, halogen, nitro, cyano, a —N═N—N(R²)₂-group or a —OSO₂Y-group,
Y denotes OH or N(R¹)₂,
R¹ denotes hydrogen, methyl or ethyl,
R² denotes methyl or ethyl;
especially, however, with the proviso that per total molecule (2) there are one to two —N═N—N(R²)₂— and one to two —OSO₂Y-groups on any ring carbon atoms of aromatic rings;
as well as its salts, hydrates, solvates and free acids and solvates of these salts and solvates of these free acids and its isomers, especially constitutional isomers, tautomers and stereoisomers, esters, prodrugs and metabolites.

According to yet another particular embodiment of the present invention according to the present first aspect of the invention, the present invention also especially relates to a diphenyl derivative for use in the prophylactic and/or therapeutic treatment of virus diseases, especially corona infections, especially COVID-19, especially a diphenyl derivative as previously defined,

wherein the diphenyl derivative corresponds to the general formula (3)

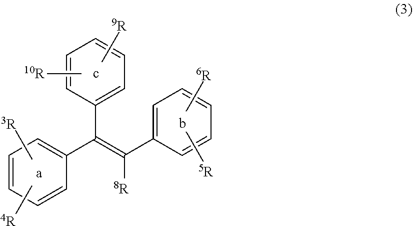

wherein:
R³, R⁶ each independently of one another denote hydrogen, hydroxyl, C₁-C₄-alkyl, C₁-C₄-alkoxy, C₁-C₄-alkyl-S—, C₁-C₄-alkyl-SO—, C₁-C₄-alkyl-SO₂—, halogen, nitro, cyano or a —OSO₂Y-group,
R⁴, R⁵ each independently of one another denote a —N═N—N(R²)₂-group or a —OSO₂Y-group,
R⁸ denotes hydrogen, ethyl, CN, NO₂, —CH₂CH₂-halogen or CH₂CH₂OH,
R⁹, R¹⁰ each independently of one another denote hydrogen, hydroxyl, C₁-C₄-alkyl, C₁-C₄-alkoxy, halogen, nitro, cyano, a —N═N—N(R²)₂-group or a —OSO₂Y-group,
Y denotes OH or N(R¹)₂,
R¹ denotes hydrogen, methyl or ethyl,
R² denotes methyl or ethyl;
especially, however, with the proviso that per total molecule (3) there are one to two —N═N—N(R²)₂-groups and one to two —OSO₂Y-groups on any ring carbon atoms of aromatic rings;
as well as its salts, hydrates, solvates and free acids and solvates of these salts and solvates of these free acids and its isomers, especially constitutional isomers, tautomers and stereoisomers, esters, prodrugs and metabolites.

According to yet another particular embodiment of the present invention according to the present first aspect of the invention, the subject matter of the present invention is especially equally a diphenyl derivative for use in the prophylactic and/or therapeutic treatment of viral diseases, especially corona infections, especially COVID-19, especially a diphenyl derivative as previously defined, wherein the diphenyl derivative corresponds to the general formula (4)

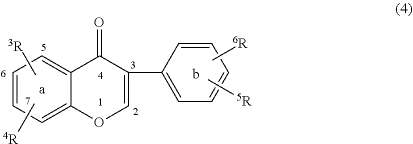

wherein
R³, R⁶ each independently of one another denote hydrogen, hydroxyl, C₁-C₄-alkyl, C₁-C₄-alkoxy, C₁-C₄-alkyl-S—, C₁-C₄-alkyl-SO—, C₁-C₄-alkyl-SO₂—, halogen, nitro, cyano or a —OSO₂Y-group,
R⁴, R⁵ each independently of one another denote a —N═N—N(R²)₂-group or a —OSO₂Y-group,
Y denotes OH or N(R¹)₂,
R¹ denotes hydrogen, methyl or ethyl,
R² denotes methyl or ethyl;
especially, however, with the proviso that per total molecule (4) there are one to two —N═N—N(R²)₂-groups and one to two —OSO₂Y-groups on any ring carbon atoms of aromatic rings;
as well as its salts, hydrates, solvates and free acids and solvates of these salts and solvates of these free acids and its isomers, especially constitutional isomers, tautomers and stereoisomers, esters, prodrugs and metabolites.

According to yet another particular embodiment of the present invention according to the present first aspect of the invention, the subject of the present invention is especially equally a diphenyl derivative for use in the prophylactic and/or therapeutic treatment of viral diseases, especially corona infections, especially COVID-19, especially a diphenyl derivative as previously defined, wherein the diphenyl derivative corresponds to the general formula (5)

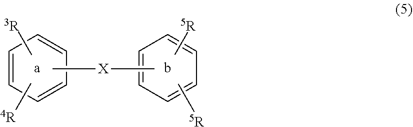

wherein
X denotes a direct C—C-bond, CH₂, CHOH, CO, S, SO, SO₂, or —N═N—,
R³, R⁶ each independently of one another denote hydrogen, hydroxyl, C₁-C₄-alkyl, C₁-C₄-alkoxy, C₁-C₄-alkyl-S—, C₁-C₄-alkyl-SO—, C₁-C₄-alkyl-SO₂—, halogen, nitro, cyano or a —OSO₂Y-group,
R⁴, R⁵ each independently of one another denote a —N═N—N(R²)₂-group or a —OSO₂Y-group,
Y denotes OH or N(R¹)₂,
R¹ denotes hydrogen, methyl or ethyl,
R² denotes methyl or ethyl;
especially, however, with the proviso that per total molecule (5) there are one to two —N═N—N(R²)₂-groups and one to two —OSO₂Y-groups on any ring carbon atoms of aromatic rings;
as well as its salts, hydrates, solvates and free acids and solvates of these salts and solvates of these free acids and its isomers, especially constitutional isomers, tautomers and stereoisomers, esters, prodrugs and metabolites.

According to yet another particular embodiment of the present invention according to the present first aspect of the invention, the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined,

wherein the diphenyl derivative, especially benzophenone derivative, corresponds to the general formula (I)

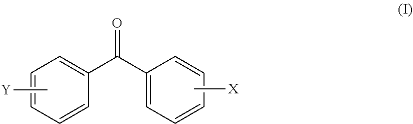

wherein:
X=represents a glucuronide-, sulfate-, triflate-, tosylate-, polyether-, carboxy-, hydroxyl-, alkoxy- and/or aryloxy-radical, and
Y=represents a nitrogen-containing radical, especially comprising a chemical group selected from amines, amides, triazines, triazenes, nitro and/or cyano;
especially with the proviso that X is not a hydroxyl radical when Y has an amine, and that X is not a sulfate when Y has a triazene;
as well as its salts, hydrates, solvates and free acids and solvates of these salts and solvates of these free acids and its isomers, especially constitutional isomers, tautomers and stereoisomers, esters, prodrugs and metabolites.

In this particular embodiment, it may be advantageous if, in the general formula (I), X is a glucuronide-, sulfate-, hydroxyl-, C₁-C₂₀-alkoxy-, especially C₁-C₁₅-alkoxy-, preferentially C₁-C₁₀-alkoxy-, and/or a phenoxy-radical, especially a glucuronide-radical, a sulfate-radical and/or a hydroxyl-radical, preferentially a glucuronide-radical or a sulfate-radical.

In this particular embodiment, it may be equally advantageous if, in the general formula (I), Y represents at least one amine of formula —NR¹R²
with R¹, R²=each independently of one another C₁-C₂₀-alkyl, C₆-C₂₀-aryl and/or hydrogen, especially C₁-C₁₀-alkyl, C₆-C₁₅-aryl and/or hydrogen, preferentially C₁-C₅-alkyl, C₆-C₁₀-aryl and/or hydrogen, preferably C₁-C₃-alkyl and/or hydrogen, more preferably hydrogen, wherein R¹ and R² are identical or different.

In this particular embodiment, it may equally also be advantageous if, in the general formula (I), Y represents at least one amide of formula —NR³C(O)OR⁴
with R³=C₁-C₂₀-alkyl, C₆-C₂₀-aryl and/or hydrogen, especially C₁-C₁₀-alkyl, C₆-C₁₃-aryl and/or hydrogen, preferentially C₁-C₅-alkyl, C₆-C₁₀-aryl and/or hydrogen, preferably C₁-C₃-alkyl and/or hydrogen, more preferably methyl and/or hydrogen, even more preferably hydrogen, and
R⁴=C₁-C₂₀-alkyl and/or C₆-C₂₀-aryl, especially C₁-C₁₀-alkyl and/or C₆-C₁₅-aryl, preferentially C₁-C₅-alkyl and/or C₆-C₁₀-aryl, preferably C₁-C₃-alkyl, more preferably methyl.

In this particular embodiment it can be equally advantageous if, in the general formula (1), Y represents at least one triazine, especially a 1,2,3-triazine, a 1,2,4-triazine and/or a 1,3,5-triazine, optionally mono- or disubstituted by C₁-C₂₀-alkyl and/or C₆-C₂₀-aryl radicals, especially C₁-C₁₀-alkyl and/or C₆-C₁₅-aryl radicals, preferentially C₁-C₅-alkyl and/or C₆-C₁₀-aryl radicals, preferably C₁-C₃-alkyl radicals.

In this particular embodiment, it may equally also be advantageous if, in the general formula (I), Y represents at least one triazene of the formula—N═N—NR⁵R⁶
with R⁵, R⁶=each independently of one another C₁-C₂₀-alkyl, C₆-C₂₀-aryl and/or hydrogen, especially C₁-C₁₀-alkyl, C₆-C₁₅-aryl and/or hydrogen, preferentially C₁-C₅-alkyl, C₆-C₁₀-aryl and/or hydrogen, preferably C₁-C₃-alkyl and/or hydrogen, more preferably methyl, wherein R⁵ and R⁶ are identical or different.

In this particular embodiment, it may equally also be advantageous,
if, in the general formula (I), X is a glucuronide radical and/or a sulfate radical, and
if, in the general formula (I), Y represents a chemical group selected from (a) amines of the formula —NR¹R² with R¹, R² each independently of one another ═C₁-C₃-alkyl and/or hydrogen, especially hydrogen; and/or (b) amides of the formula —NR³C(O)OR⁴ with R³=C₁-C₃-alkyl, especially methyl, and/or hydrogen, preferably hydrogen, and R⁴=C₁-C₃-alkyl, especially methyl; and/or (c) triazenes of the formula —N═N—NR⁵R⁶ with R⁵, R⁶ each independently of one another C₁-C₃-alkyl and/or hydrogen, especially methyl,
especially, however, with the proviso that X is not a sulfate if Y is a triazene.

According to yet another particular embodiment of the present invention according to the present first aspect of the invention, the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined,

wherein the diphenyl derivative, especially benzophenone derivative, is a diphenyl derivative represented by the following formulae (II-A) to (II-F):

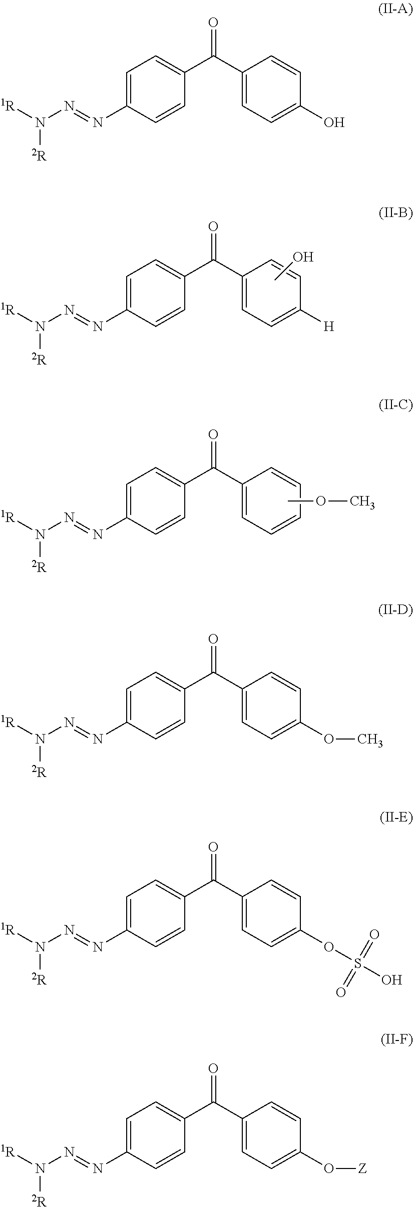

wherein:
R¹ and R² each independently of one another denote optionally substituted alkyl, especially optionally substituted C₁-C₂₀-alkyl, optionally substituted alkenyl, especially optionally substituted C₂-C₂₀-alkenyl, or optionally substituted aryl, especially optionally substituted C₆-C₂₀-aryl, and
Z denotes a sugar radical or a glucuronide radical;
as well as its salts, hydrates, solvates and free acids and solvates of these salts and solvates of these free acids and its isomers, especially constitutional isomers, tautomers and stereoisomers, esters, prodrugs and metabolites.

According to yet another particular embodiment of the present invention according to the present first aspect of the invention, the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined, wherein the diphenyl derivative, especially benzophenone derivative, is a diphenyl derivative represented by the following formulae (III-A) to (III-D):

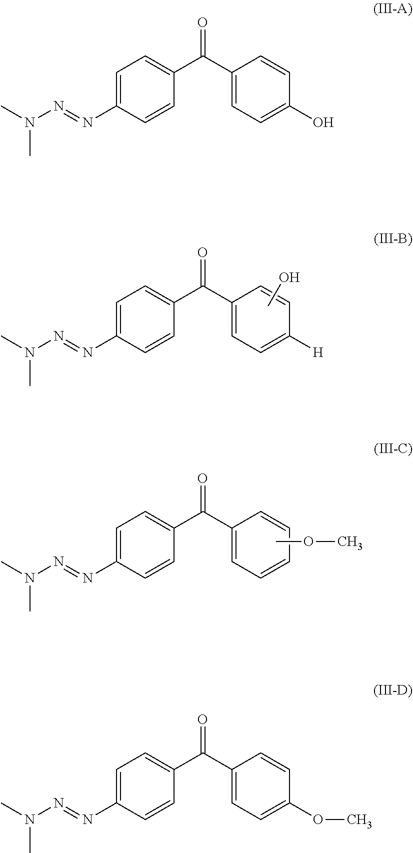

as well as its salts, hydrates, solvates and free acids and solvates of these salts and solvates of these free acids and its isomers, especially constitutional isomers, tautomers and stereoisomers, esters, prodrugs and metabolites.

According to yet another particular embodiment of the present invention according to the present first aspect of the invention, the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined, wherein the diphenyl derivative, especially benzophenone derivative, is a diphenyl derivative represented by the following formulae (IV-A) to (IV-I):

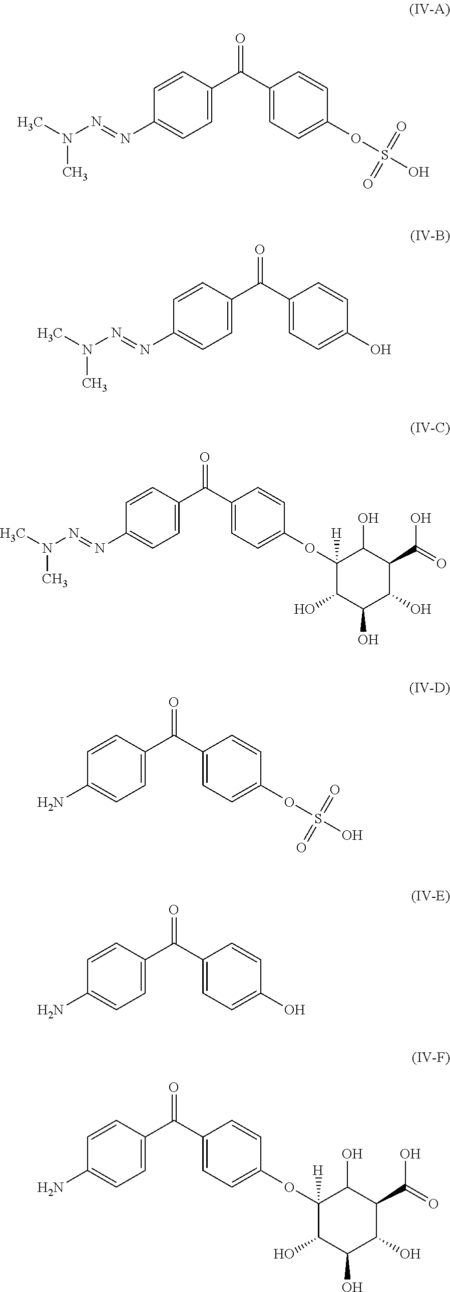 [SMILES: CN(C)/N=N/c1ccc(C(=O)c2ccc(OS(=O)(=O)O)cc2)cc1 ; CN(C)/N=N/c1ccc(C(=O)c2ccc(O)cc2)cc1 ; CN(C)/N=N/c1ccc(C(=O)c2ccc(O[C@H]3C(O)[C@@H](C(=O)O)[C@H](O)[C@@H](O)[C@@H]3O)cc2)cc1 ; Nc1ccc(C(=O)c2ccc(OS(=O)(=O)O)cc2)cc1 ; Nc1ccc(C(=O)c2ccc(O)cc2)cc1 ; Nc1ccc(C(=O)c2ccc(O[C@H]3C(O)[C@@H](C(=O)O)[C@H](O)[C@@H](O)[C@@H]3O)cc2)cc1]

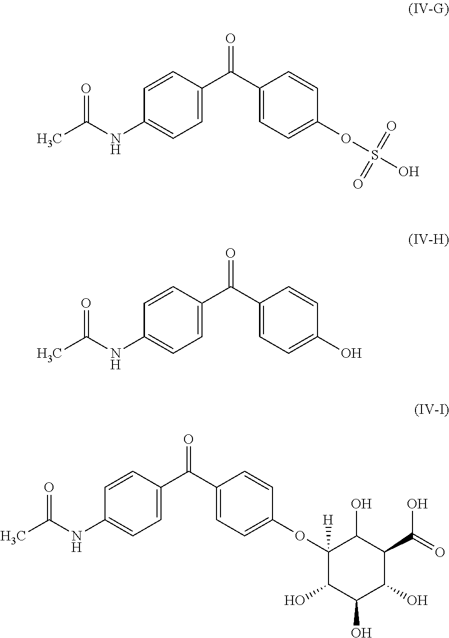 [SMILES: CC(=O)Nc1ccc(C(=O)c2ccc(OS(=O)(=O)O)cc2)cc1 ; CC(=O)Nc1ccc(C(=O)c2ccc(O)cc2)cc1 ; CC(=O)Nc1ccc(C(=O)c2ccc(O[C@H]3C(O)[C@@H](C(=O)O)[C@H](O)[C@@H](O)[C@@H]3O)cc2)cc1]

as well as its salts, hydrates, solvates and free acids and solvates of these salts and solvates of these free acids and its isomers, especially constitutional isomers, tautomers and stereoisomers, esters, prodrugs and metabolites.

According to yet another particular embodiment of the present invention according to the present first aspect of the invention, the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined,

wherein the diphenyl derivative, especially benzophenone derivative, is a diphenyl derivative represented by the following formula (V-A):

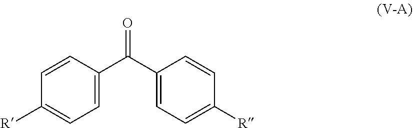

wherein:
R′ denotes a radical (CH₃)₂N—N═N—, a radical CH₃—C(O)—NH— or a radical NH₂— and preferentially denotes a radical (CH₃)₂N—N═N—,
R″ denotes one of the following radicals:
   —OH
   —O—SO₂—OH
   —O—CH₂—C(O)OH
   —O—CH₂—C(O)OR″ with R″=C₁-C₄-alkyl, glycinyl, glucuronide radical
   —O—CH₂—CH₂—C(O)—NH—CH₂—C(O)—O(C₁-C₄-alkyl)
   —O—CH₂—CH₂—C(O)—NH—CH₂—C(O)—OH
   —O—CH₂—CH₂—C(O)—OH
   —O—CH₂—CH₂—C(O)—OR″ with R″═C₁-C₄-alkyl, glycinyl, glucuronide radical

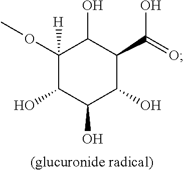

as well as its salts, hydrates, solvates and free acids and solvates of these salts and solvates of these free acids and its isomers, especially constitutional isomers, tautomers and stereoisomers, esters, prodrugs and metabolites.

According to yet another particular embodiment of the present invention according to the present first aspect of the invention, the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined, wherein the diphenyl derivative, especially benzophenone derivative, is a diphenyl derivative represented by the following formula (IV-A1) or (IV-A2):

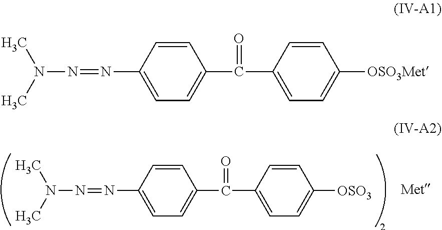

wherein:
Met′ denotes a monovalent metal ion, especially an alkali metal ion, preferentially a sodium ion, and
Met″ denotes a divalent metal ion, especially an alkaline earth metal ion.

According to yet another particular embodiment of the present invention according to the present first aspect of the invention, the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined,

wherein the diphenyl derivative, especially benzophenone derivative, is a diphenyl derivative represented by the following formula (IV-A3):

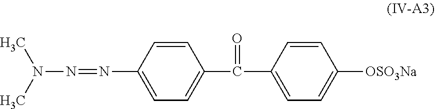

According to yet another particular embodiment of the present invention according to the present first aspect of the invention, the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined,

wherein the diphenyl derivative, especially benzophenone derivative, is a diphenyl derivative represented by the following formula (IV-A):

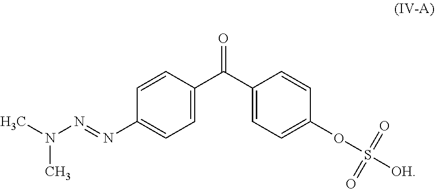 [SMILES: CN(C)/N=N/c1ccc(C(=O)c2ccc(OS(=O)(=O)O)cc2)cc1]

According to yet another particular embodiment of the present invention according to the present first aspect of the invention, the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined, wherein the diphenyl derivative, especially benzophenone derivative, is a diphenyl derivative represented by the following formula (VI-A):

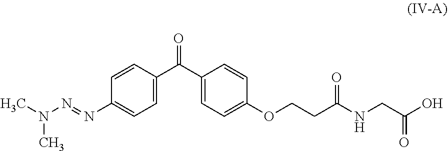 [SMILES: CN(C)/N=N/c1ccc(C(=O)c2ccc(OCCC(=O)NCC(=O)O)cc2)cc1]

as well as its salts, hydrates, solvates and free acids and solvates of these salts and solvates of these free acids and its isomers, especially constitutional isomers, tautomers and stereoisomers, prodrugs and metabolites, as well as its esters, preferentially C₁-C₄-alkyl esters, glycinyl esters or glucuronide esters (i.e. esters at the right free hydroxyl of the above formula).

Furthermore—according to yet another particular embodiment of the present invention according to the present first aspect of the invention—the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined, wherein the diphenyl derivative, especially benzophenone derivative, is a diphenyl derivative represented by the following formula (VII-A):

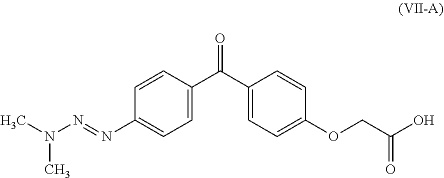 [SMILES: CN(C)/N=N/c1ccc(C(=O)c2ccc(OCC(=O)O)cc2)cc1]

as well as its salts, hydrates, solvates and free acids and solvates of these salts and solvates of these free acids and its isomers, especially constitutional isomers, tautomers and stereoisomers, prodrugs and metabolites as well as its esters, preferentially C₁-C₄-alkyl esters, glycinyl esters or glucuronide esters (i.e. esters at the right free hydroxyl of the above formula).

Finally—according to yet another particular embodiment of the present invention according to the present first aspect of the invention—the subject matter of the present invention is especially equally a diphenyl derivative, especially benzophenone derivative, for use in the prophylactic and/or therapeutic treatment of viral diseases, especially corona infections, especially COVID-19, especially a diphenyl derivative as previously defined, wherein the diphenyl derivative, especially benzophenone derivative, is a diphenyl derivative represented by the following formula (VIII-A):

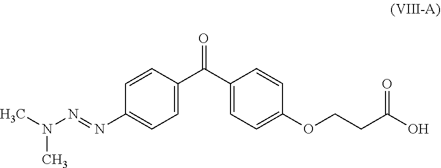 [SMILES: CN(C)/N=N/c1ccc(C(=O)c2ccc(OCCC(=O)O)cc2)cc1]

as well as its salts, hydrates, solvates and free acids and solvates of these salts and solvates of these free acids and its isomers, especially constitutional isomers, tautomers and stereoisomers, prodrugs and metabolites as well as its esters, preferentially C₁-C₄alkyl esters, glycinyl esters or glucuronide esters (i.e. esters at the right free hydroxyl of the above formula).

With regard to the first aspect of the invention, especially with regard to all the embodiments described above, it should be stated especially that the diphenyl derivative is in each case in the form of its physiologically tolerable salts, free acids, solvates or solvates of these salts or solvates of these free acids, hydrates or isomers, especially constitutional isomers, tautomers and stereoisomers, derivatives, esters, prodrugs and metabolites, preferentially in the form of its physiologically tolerable salts, more preferably in the form of an alkali metal or alkaline earth metal salt.

With regard to the first aspect of the invention, especially with regard to all embodiments described above, it is to be further stated especially that the diphenyl derivative is preferably applied and used systemically.

With regard to the first aspect of the invention, especially with regard to all the embodiments described hereinabove, it should also be stated especially that optionally the diphenyl derivative, especially the drug or medicament containing the diphenyl derivative, can be used or applied together with at least one further active ingredient, wherein the at least one further active ingredient can be selected especially from anti-inflammatory active ingredients (especially corticosteroids, such as, for example, dexamethasone), blood-thinning active ingredients with antiviral activity and combinations thereof.

A further subject matter of the present invention—according to a second aspect of the present invention—is the inventive use of a diphenyl derivative, as defined hereinabove, for the prophylactic and/or therapeutic treatment of viral infections, especially corona infections, especially COVID-19, or the inventive use of the above-mentioned active ingredients or diphenyl derivatives for the preparation of a drug or medicament for the prophylactic and/or therapeutic treatment of viral infections (virus infections), especially infections with corona viruses, especially COVID-19.

In the context of the inventive use according to the present aspect of the invention, it is particularly intended that the active ingredient or the diphenyl derivative is applied systemically.

Furthermore, within the scope of the use according to the invention according to this aspect of the invention, it may be intended, if necessary, that the active ingredient or the diphenyl derivative is used or applied together with at least one further active ingredient. The further active ingredient may especially be selected from anti-inflammatory active ingredients (especially corticosteroids, such as dexamethasone), blood-thinning active ingredients, active ingredients with antiviral activity, and combinations thereof. Especially, the other active ingredient is also used or applied systemically.

For further details on this aspect of the invention, reference may be made to the preceding explanations concerning the first aspect of the invention, which explanations apply equally to the present aspect of the invention.

Equally a subject matter of the present invention—according to a third aspect of the present invention—is the inventive use of a diphenyl derivative, as previously defined, as an antiviral agent for the prophylactic and/or therapeutic treatment of viral diseases, especially corona infections, especially COVID-19, or for the preparation of an antiviral agent for the prophylactic and/or therapeutic treatment of viral diseases, especially corona infections, especially COVID-19.

In the context of the inventive use according to the present aspect of the invention, it is particularly intended that the active ingredient or the diphenyl derivative is applied systemically.

Furthermore, within the scope of the inventive use according to this aspect of the invention, it may be intended, if necessary, that the active ingredient or the diphenyl derivative is used or applied together with at least one further active ingredient. The further active ingredient may especially be selected from anti-inflammatory active ingredients (especially corticosteroids, such as dexamethasone), blood-thinning active ingredients, active ingredients with antiviral activity, and combinations thereof. Especially, the other active ingredient is also used or applied systemically.

For further details on this aspect of the invention, reference may be made to the preceding discussion of the aspects of the invention, which discussion applies equally to the present aspect of the invention.

Yet another subject matter of the present invention—according to a fourth aspect of the present invention—is a method according to the invention for the prophylactic and/or therapeutic treatment of viral diseases, especially corona infections, especially COVID-19, wherein in the method a pharmaceutically effective or therapeutically effective amount of a diphenyl derivative, as previously defined, is administered to a patient.

Within the scope of the method according to the present aspect of the invention, it is particularly intended that the active ingredient or the diphenyl derivative is applied systemically.

Furthermore, within the scope of the method according to the present aspect of the invention, it may be provided, if necessary, that the active ingredient or the diphenyl derivative is used or applied together with at least one further active ingredient. The further active ingredient may especially be selected from anti-inflammatory active ingredients (especially corticosteroids, such as dexamethasone), blood-thinning active ingredients, active ingredients with antiviral activity, and combinations thereof. Especially, the other active ingredient is also used or applied systemically.

For further details on this aspect of the invention, reference may be made to the preceding discussions of the aspects of the invention, which discussions apply equally to the present aspect of the invention.

Also a subject matter of the present invention—according to a fifth aspect of the present invention—is a drug or medicament for (use in the) prophylactic and/or therapeutic treatment of viral diseases, especially corona infections, especially COVID-19, especially antiviral agents, wherein the drug or medicament, especially antiviral agent, comprises at least one diphenyl derivative, as previously defined, together with a pharmaceutically acceptable carrier or excipient.

Also within the scope of this aspect of the invention, it is particularly envisaged that the active ingredient or diphenyl derivative is applied systemically.

Furthermore, within the scope of the inventive use according to this aspect of the invention, it may be provided, if necessary, that the active ingredient or the diphenyl derivative is used or applied together with at least one further active ingredient. The further active ingredient may especially be selected from anti-inflammatory active ingredients (especially corticosteroids, such as dexamethasone), blood-thinning active ingredients, active ingredients with antiviral activity, and combinations thereof. Especially, the other active ingredient is also used or applied systemically.

For further details on this aspect of the invention, reference may be made to the preceding discussions of the aspects of the invention, which discussions apply equally to the present aspect of the invention.

Finally, it is equally a subject matter of the present invention—according to a sixth aspect of the present invention—to provide a pharmaceutical combination for (use in the) prophylactic and/or therapeutic treatment of viral diseases, especially corona infections, especially COVID-19, where the pharmaceutical combination comprises
(A) at least one diphenyl derivative as defined above or as defined in any of claims 1 to 21; and
(B) at least one further active ingredient selected from anti-inflammatory active ingredients, especially corticosteroids, blood-thinning active ingredients, antiviral active ingredients as well as combinations thereof.

Also within the scope of this aspect of the invention, it is particularly envisaged that the active ingredient or diphenyl derivative is applied systemically.

The other active ingredient is especially also used or applied systemically in the same way.

For further details on this aspect of the invention, reference may be made to the preceding discussions of the aspects of the invention, which discussions apply equally to the present aspect of the invention.

The active ingredients used according to the invention or the diphenyl derivatives used according to the invention (as defined above) also possess especially a general (i.e. universally valid) or comprehensive or virus nonspecific antiviral action potential and/or efficacy spectrum. Consequently, the active ingredients used according to the invention or the diphenyl derivatives used according to the invention can be used to treat virtually all types of viral diseases. This applies to all previously described aspects of the invention (i.e. first through sixth aspects of the present invention, as previously described). Thus, according to all aspects of the invention described above, the active ingredients used according to the invention or the diphenyl derivatives used according to the invention (as defined above) can be used or employed for the prophylactic and/or therapeutic treatment of viral diseases, wherein the viral diseases may especially be selected from the group of corona infections, such as MERS and SARS, especially COVID-19, HIV-infections, influenza, parainfluenza, cytomegalovirus infections (HCMV-infections), papillomavirus infections (HPV-infections), viral hepatitides (virus hepatitides), enterovirus infections, Epstein-Barr virus infections, herpes virus infections, varicella virus infections, especially varicella zoster virus infections, adenovirus infections, rotavirus infections, yellow fever virus infections, viral hemorrhagic fever (VHF), TBE virus infections, West-Nile virus infections, ebola virus infections, rabies (rabies), marburg virus infections, hantavirus infections, lassa virus infections, dengue virus infections, arena virus infections, arbovirus infections, coxsackie virus infections, measles, mumps, rubella and ringworm rubella virus infections, and poliovirus infections, preferentially from the group of corona infections, especially COVID-19, HIV-infections, influenza, parainfluenza, cytomegalovirus infections (HCMV-infections), papillomavirus infections (HPV-infections) and viral hepatitis (virus hepatitis), more preferably from the group of corona infections, especially COVID-19.

Further embodiments, modifications and variations as well as advantages of the present invention are readily apparent and realizable to those skilled in the art upon reading the description, without departing from the scope of the present invention.

The following embodiments are merely illustrative of the present invention, but without limiting the present invention thereto.

### EXAMPLES

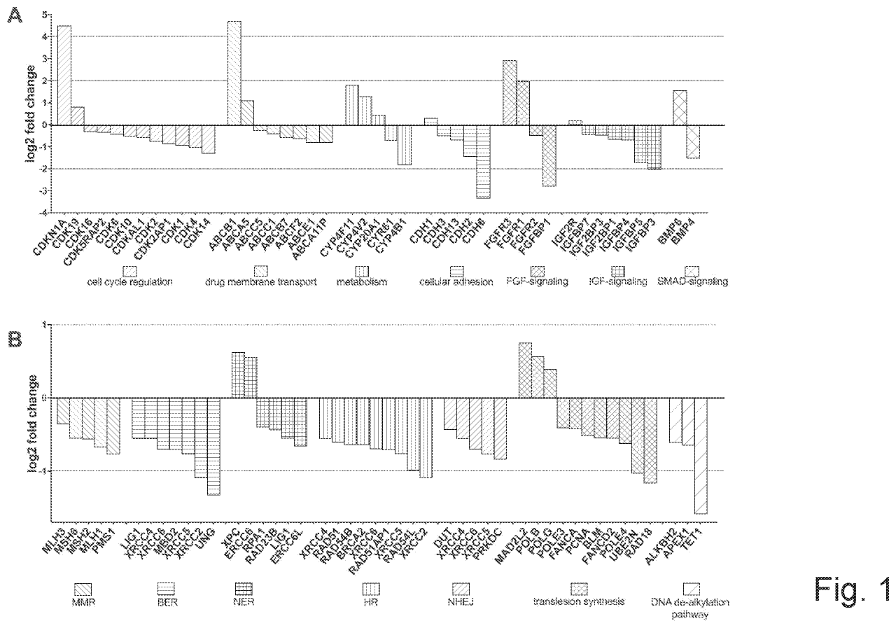

1. Shutdown of Cellular Repair Systems as a New Approach for Antiviral Therapeutics
In the context of the present invention, it has been surprisingly found that the diphenyl derivatives used in accordance with the invention have an antiviral effect. Without wishing to limit ourselves to this theory, it is assumed that the antiviral mode of action is based on a downregulation of certain cellular repair systems.
In this context, first experiments were carried out with a diphenyl derivative according to the above formula (IV-A3) (wherein this diphenyl derivative, which is equally a triazene, is hereinafter simply referred to as “compound (IV-A3)”) and with a diphenyl derivative according to the above formula (III-A) (wherein this diphenyl derivative, which is also a triazene, is hereinafter simply referred to as “compound (III-A)”).
In addition, further experiments were carried out with the diphenyl derivatives according to the structures of claims 13, 14, 16, 18 and 19, which give substantially the same results.
For the sake of simplicity, however, only the experimental results for compound (IV-A3) and compound (III-A) are reported below.
Compound (IV-A3) strongly induced CDKN1A (cyclin dependent kinase inhibitor 1A) transcription, suggesting cell cycle blockade as the first molecular mode of action. In addition, the compound (compound (IV-A3)) decreased the expression of certain gene segments involved in the regulation of certain DNA repair mechanisms, such as homologous recombination (HR), dealkylation of DNA, mismatch repair (MMR), and nucleotide excision repair (NER), respectively.
RNA isolation and whole transcriptome sequencing:
For total transcriptome sequencing (RNA-Seq), different approaches of cells (3.5×10-6 cells UWB1.289 or 3.3×10-6 cells UWB1.289+BRCA1) were grown in T75 culture vessels. After 24 hours, the medium was removed and replaced with new medium containing 1,000 μM of compound (IV-A3) or 40 μM of compound (III-A). Standard culture medium served as a control. After 24 h of substance exposure, cells were harvested and RNA was extracted using the standard miRNeasy procedure (Qiagen, Hilden, Germany). To remove contaminations with residual genomic DNA, a column purification step was performed. All experiments were performed in triplicate and were evaluated according to existing manufacturer's instructions. For gene library construction, the TruSeq Stranded mRNA Lbrary Prep Kit (Illumina) was used according to the manufacturer's instructions, starting with 1 sg of total RNA. The encoded libraries (barcoded) were pooled and sequenced on an Illumina NextSeq500 platform.
Bioinformatic analysis of transcriptome results:
The evaluation was performed using Trimmomatic and STAR software. Hierarchical clustering was performed using the so-called Euclid distance and association formation and visualized with Complex Heatmap. Furthermore, principal component analysis (principal component analysis or PCA plot) was performed using the R Stats package from the company R-Institut für statistisches Rechnen, Vienna, Austria. The DESeq2 method was applied to identify differentially expressed genes according to standard criteria. Only genes with multiply corrected p-values less than 0.05 (padj of DESeq2) were considered significant.
Transcriptional profiles of compound (IV-A3) and compound (III-A) treated ovarian cancer cells:
To investigate the mechanism of action of diphenyl derivatives or triazenes used according to the invention in ovarian cancer, a comprehensive gene expression analysis was performed in UWB1,289 cell pairs treated with compound (IV-A3) and compound (III-A), respectively. Samples of BRCA1-deficient and BRAC1-proficient UWB1,289 cells were subjected to short-term treatment in duplicate (1,000 μM compound (IV-A3) or 40 μM compound (III-A) for 24 hours). At the time of RNA isolation and transcriptome sequencing, no signs of morphological changes were found on the cells. It was found that hierarchical grouping and principal component analysis revealed a clear difference in transcriptome profiles between the two cell lines and depending on the drugs used (compound (IV-A3) and compound (III-A), respectively). In addition, cells treated with compound (IV-A3) showed greater differences from non-treated controls in the PCA plot compared to cells treated with compound (III-A). This indicates that compound (IV-A3) induces more pronounced changes in the transcriptome than compound (III-A). Nonetheless, regardless of the differences, both compounds induce sufficient change in the transcriptome to assume a therapeutic effect. This finding was further supported by the fact that a higher number of differentially transcribed genes were found in compound (IV-A3) compared to compound (III-A) and the control cells.
In addition, differential gene expression analysis was performed with a focus on those genes involved in the regulation of the cell cycle, drug-membrane transport, metabolism, cellular adhesion, and other disease-related metabolic pathways, such as fibroblast growth factor, short FGF, and the SMAD pathway, respectively). The intersections of genes differentially expressed in both cell lines after compound treatment were determined (log 2FC values).
Treatment with compound (IV-A3) results in marked down regulation of several CDK family genes and concomitant strong induction of CDKN1A (log 2FC=4.49).
This suggests activation of the cyclin-dependent protein kinase inhibitor pathway and arrest of the cell cycle as an early response to treatment. Consistent with the enhanced cell detachment after treatment with compound (IV-A3), some cadherin cell adhesion molecule genes, particularly CDH6, were downregulated (compare FIG. 1A).
In addition to certain gene regulatory effects of compound (IV-A3) and compound (III-A) on the expression of drug efflux pump mechanisms (ABC transporter family) and on some cytochrome enzymes, it was found that a number of DNA repair systems especially, e.g mismatch repair (MMR), base excision repair (BER; XRCC2), nucleotide excision repair (NER; ERCC6) or DNA damage signaling sensory systems (ATM u. ATR kinases) or homologous recombination repair (HR; XRC2), are down-regulated at the transcription level (log 2FC=−1.09). Notably, the major proteins of the DNA dealkylation pathway (APEX1, TET1, or ALKBH2), which play important roles in triazene-mediated DNA dealkylation, were consistently downregulated after treatment with compound (IV-A3) in both cell lines (compare FIG. 1B). Treatment with compound (IV-A3) or compound (III-A) also leads to consistent upregulation of CDKN1A, accompanied by downregulation of CDK proteins and essential DNA repair enzymes. Compound (IV-A3) also induces certain changes at the transcriptome level, which play a role in the regulation of cell cycle, membrane transport, drug metabolism and cell adhesion. In addition, compound (IV-A3) causes extensive downregulation of transcripts that play functionally important roles in various DNA repair systems.
FIG. 1A and FIG. 1B show purely exemplary transcription patterns of compound (IV-A3)-treated UWB1,289 ovarian cancer cells using waterfall plots:
FIG. 1A shows the differentially regulated gene expression of genes involved in cell cycle regulation, drug membrane transport, drug metabolism, cell adhesion and other selected metabolic pathways in UWB1,289 cells after treatment with compound (IV-A3). FIG. 1B shows differential gene expression of genes involved in metabolic pathways regulating DNA repair in UWB1,289 cells after treatment with compound (IV-A3).
From the transcriptional analyses described above, it can be deduced that the compounds based on diphenyl derivatives or triazenes used in accordance with the invention have a significant influence on the gene expression of genes that are involved in various DNA repair mechanisms, but also genes that are involved in cell cycle regulation, drug membrane transport, drug metabolism, cell adhesion and other selected metabolic pathways. Without wishing to be limited to this theory, it is assumed in the context of the present invention that especially the influence on the expression of genes which regulate or coordinate the cellular repair systems is at the same time the basis for the antiviral efficacy of the compounds based on diphenyl derivatives or triazenes used according to the invention.

2. Investigation of the Antiviral Activity of Compounds According to the Invention
In order to investigate the antiviral activity of the compound of the invention, the experiments described below were carried out.
The further experimental tests were carried out with the following compounds:
   a compound of the above formula (VI-A), hereinafter also referred to simply as “compound (VI-A)” or “(VI-A)” (used in the form of the sodium salt);
   a compound of the above formula (VIII-A), hereinafter also referred to simply as “compound (VIII-A)” or “(VIII-A)”;
   a compound of the above formula (IV-A), hereinafter also referred to simply as “compound (IV-A)” or “(IV-A)”;
   a compound of the above formula (IV-B), hereinafter also referred to simply as “compound (IV-B)” or “(IV-B)”.

Studies of the Therapeutic Effect in the Mouse Model
To investigate the therapeutic effect of compound (VI-A) against SARS-CoV-2 infection in mouse model, K18-hACE2 transgenic mice were used. The viruses used were a German isolate of SARS-CoV-2. The study was conducted as a feasibility study with two groups (test group, control group) based on six mice each, i.e. a total of 12 animals. The two groups of mice were mildly anesthetized by inhalation of 3% isoflurane and intranasally inoculated with 300 KWSIU (FFU) of SARS-CoV-2. Treatment with the compound (VI-A) was performed by intra-peritoneal injection or application. Treatment of the first group began 30 minutes before infection with a compound concentration or dosage of 150 mg/kg. In addition, three hours after infection, the test mice were administered another 500 mg/kg of the compound (dissolved in 100 μl total volume of simply concentrated PBS buffer with pH 7.4). In the second group (positive control or control group), the mice were infected equally but treated with PBS buffer alone without the addition of diphenyl derivatives or triazenes (sham treatment). Two days post infection, the animals were euthanized. The clinical score (“clinical score”), comprising the recording of weight and clinical signs, of the animals was determined. To determine the success of therapy, the viral load in the lungs was determined by qPCR. In addition, immuno-histochemical and histopathological examinations of the lungs were performed, namely lung histology based on hematoxylin-eosin staining and immunostaining for the detection of SARS-CoV-2 virus.
In qPCR, it has been shown that the viral load in the lungs of the compound (VI-A) treated animals was significantly lower compared to the positive control group. In addition, histological examinations of the lung tissue also showed a significantly reduced incidence of infection compared to the control group.
The experiments were further performed or repeated with the other compounds (VIII-A), (IV-A) and (IV-B). Equally excellent results were obtained with these compounds, suggesting a significant antiviral effect, due to the significantly reduced viral load in the lungs after treatment of SARS-CoV-2 infection with the aforementioned compounds.

Investigation of Antiviral Mode of Action Using icELISA
An icELISA (in-cell ELISA) was used to determine the amount of viral SARS-CoV-2 N- or S-proteins in infected cells after treatment with compounds according to the invention. Compound (VIII-A), compound (VI-A), compound (IV-A), and compound (IV-B) were used for the respective studies. For the icELISA studies, human Caco-2 cells were used on the one hand and Vero-E6 cells on the other.
The experimental setup is described below.

Cells, Viruses and Infection
For ELISA studies, Vero-E6 (ATCC CRL-1586) and Caco-2 (ATCC HTB-37) cells were first cultured in Dulbecco's Modified Eagle's Medium (DMEM) with high glucose concentration, and the medium was supplemented with 10% fetal calf serum (FCS), penicillin, and streptomycin. Cultivation was performed at 37° C. at atmospheric pressure with 5% CO₂. SARS-CoV-2 strains for subsequent cell infection were isolated from patients, with isolation and virus propagation in Vero-E6 cells. Isolation of SARS-CoV-2 viruses was confirmed diagnostically by qRT-PCR. Viral titers were determined by CCID50 titration.

Diphenyl Derivatives or Triazenes Used
For the following icELISA, solutions and dilutions of compound (VI-A), compound (VIII-A), compound (IV-A) and compound (IV-B) were prepared. The concentration data and the sol-vents used in each case can be taken from the following table:

| | Stock | | | | | |
|---|---|---|---|---|---|---|
| compound | solutions | Medium | 1/4000 | 1/2000 | 1/1000 | 1/500 |
| (VI-A) | 200 | H₂O | 50 | 100 | 200 | 400 |
| | mg/ml | | μg/ml | μg/ml | μg/ml | μg/ml |
| (VIII-A) | 500 | DMSO | 125 | 250 | 500 | 1000 |
| | mg/ml | | μg/ml | μg/ml | μg/ml | μg/ml |
| (IV-A) | 100 | H₂O | 25 | 50 | 100 | 200 |
| | mg/ml | | μg/ml | μg/ml | μg/ml | μg/ml |
| (IV-B) | 100 | DMSO | 25 | 50 | 100 | 200 |
| | mg/ml | | μg/ml | μg/ml | μg/ml | μg/ml |

The dilutions 1/4000, 1/2000, 1/1000 and 1/500 specified in the table were subsequently used to treat the cells for icELISA

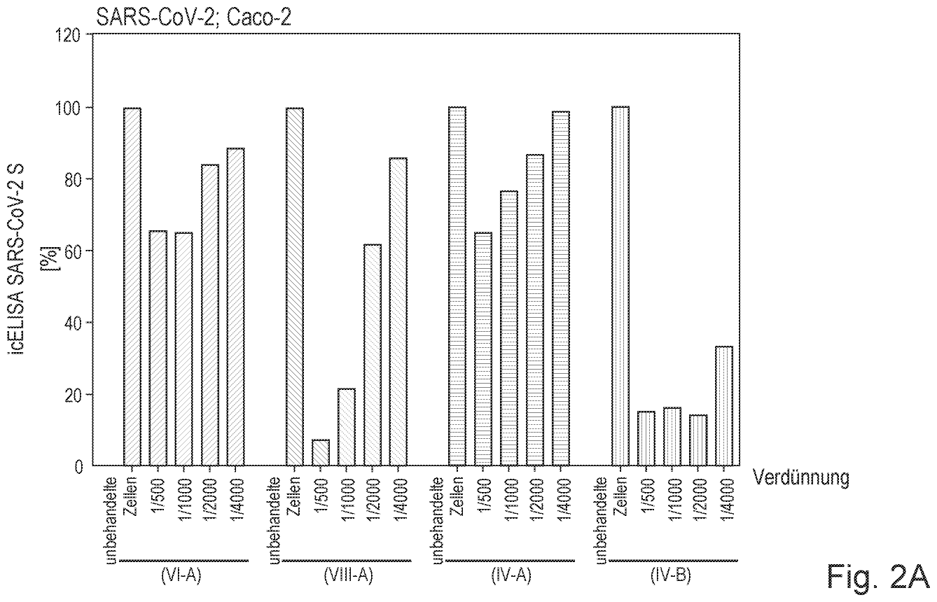

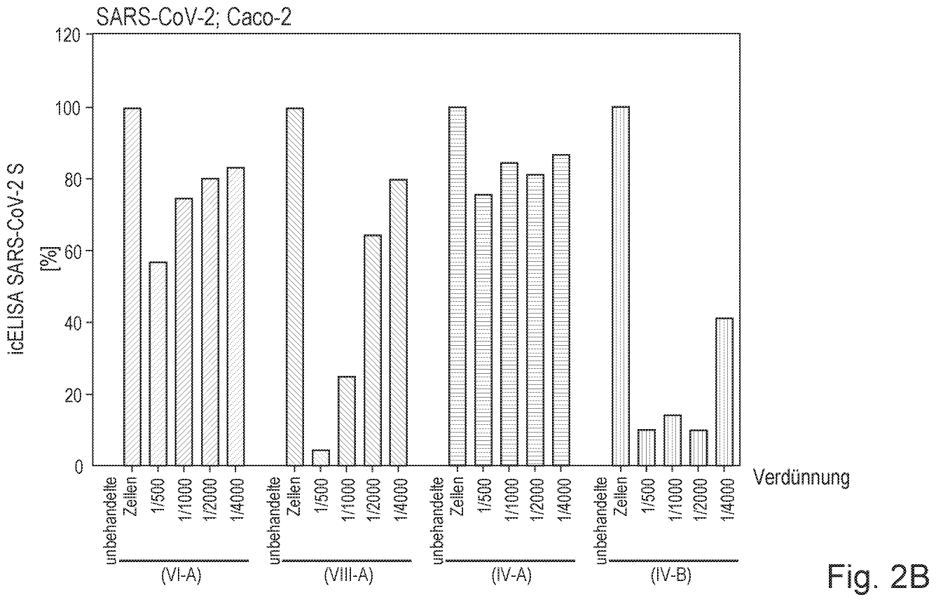

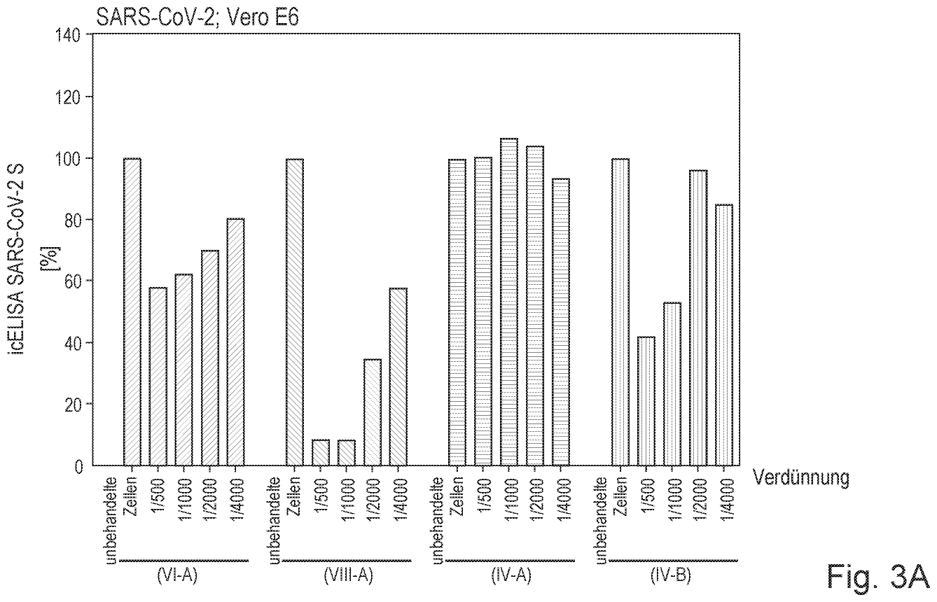

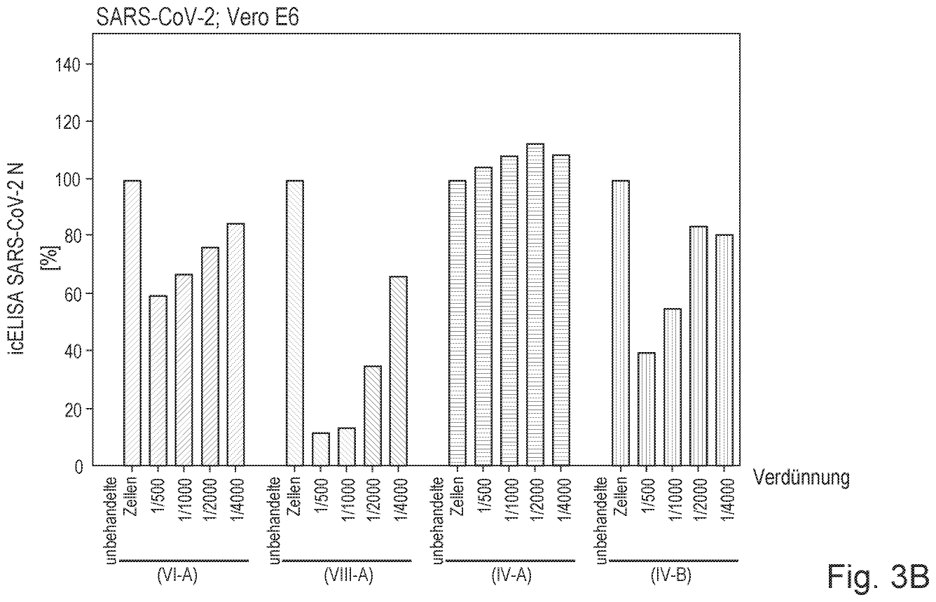

In-Cell ELISA (icELISA)
To quantify the amount of viral SARS-CoV-2 proteins in infected cells, icELISA was performed. Caco-2 cells and Vero-E6 cells were used for the ELISA studies, and two approaches were measured for each cell type. ELISA was performed according to the protocol described in Scholer et al: “A Novel In-Cell ELISA Assay Allows Rapid and Automated Quantification of SARS-CoV-2 to Analyze Neutralizing Antibodies and Antiviral Compounds,” Front Immunol 11, 573526 (2020). Before performing icELISA, cells were infected with SARS-CoV-2 and fixed with 4% w/v (weight/volume) paraformaldehyde in PBS 20 hours after infection to perform ELISA. Cells were permeabilized with 1% by volume Triton X-100 in PBS and blocked with 3% by volume fetal calf serum in PBS. The first antibody was then added and incubated for two hours at room temperature or overnight at 4° C. The batch was then incubated with a peroxidase-labeled secondary antibody for one to two hours. This was followed by a wash step with 0.05% by volume Tween-20 in PBS. To visualize the enzyme reaction, tetramethylbenzidine was added as a substrate. The reaction was stopped with 0.5 M HCl before absorbance was determined using a microplate reader and appropriate software was evaluated.
The icELISA results obtained for the two approaches with Caco-2 cells are shown in FIGS. 2A and 2B. The results obtained with Vero-E6 cells are shown in FIGS. 3A and 3B.
The amount of viruses in the cells was quantified in the ELISA For this purpose, the viral load measured in untreated cells was set as a reference value of 100% in the subsequent evaluation. It was determined how the viral load changes in percent compared to the reference value for untreated cells with the different compounds in different concentrations.
FIGS. 2A and 2B show the mean values from each of three independent experiments with SARS-CoV-2 infected Caco-2 cells. It is evident from both FIG. 2A and FIG. 2B that a significant decrease in viral load was achieved in treated cells compared to untreated cells with all compounds. Particularly good results were obtained with compounds (VIII-A) and (IV-B). For compound (IV-B), even with the strongest dilution, i.e. the lowest drug concentration, a decrease in viral load of 50% and 40%, respectively, was still achieved. Overall, the best results in terms of viral load decrease were obtained with compound (VIII-A) at a dilution of 1/500 (see table above).
Similar observations were made in icELISA results with SARS-CoV-2 infected Vero-E6 cells (compare FIG. 3A and FIG. 3B). Here, the greatest decrease in viral load was also obtained with compound (VIII-A). With compound (VI-A) and compound (IV-B), a significant decrease in viral load was equally obtained in Vero-E6 cells as a result of treatment.

The icELISA results show that the diphenyl derivatives or triazenes used according to the invention lead to a significant overall decrease in SARS-CoV-2 viral load in infected cells.
Overall, the diphenyl derivatives or triazenes according to the invention therefore represent a promising starting point for use as therapeutics for the treatment of viral diseases, especially viral diseases triggered by corona viruses.

Conclusion and Outlook
In view of the fact that the infection pathways of various viruses and the mechanisms by which different viruses attack especially human cells are sometimes similar, it can be assumed that the diphenyl derivatives or triazenes according to the invention are suitable not only for the treatment of infections with corona viruses, but also for the treatment of infections with other types of viruses, especially RNA viruses. Especially, the diphenyl derivatives or triazenes according to the invention also appear to be a suitable starting point for the treatment of infections with HI-viruses. An initial efficacy of the compounds (VI-A), (VIII-A), (IV-A) and (IV-B) according to the invention was determined by EASY-HIT assay. Here, a first inhibitory effect on HI-viruses could already be detected.

1-30. (canceled)

31. A medicament for the treatment of a viral disease selected among corona infections,
wherein the medicament comprises, together with a pharmaceutically acceptable, at least one antiviral agent comprising at least one diphenyl derivative including its salts, hydrates, solvates, free acids or solvates of these salts or solvates of these free acids or isomers, esters, prodrugs and metabolites,
wherein the diphenyl derivative comprises per molecule
(i) at least one dialkyltriazenyl group and
(ii) at least one further group being different from (i) and being selected among sulfooxy groups and sulfamoyloxy groups.

32. The medicament according to claim 31,
wherein the viral disease is COVID-19.

33. The medicament according to claim 31,
wherein the diphenyl derivative corresponds to the general formula (1) 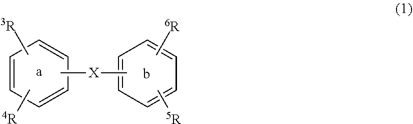
wherein:
X denotes a direct C—C bond between the rings a and b, CH₂, CHOH, CO, S, SO, SO₂, —N═N—, —CR⁷═CR⁸— or a divalent —C(O)—C*═CH—O— radical which, together with the two adjacent C-atoms of the ring a on which it is located, forms a pyranone ring, the ring b being located on the C* atom of this radical,
R³, R⁶ each independently of one another denote hydrogen, hydroxyl-, C₁-C₄-alkyl, C₁-C₄-alkoxy, C₁-C₄-alkyl-S—, C₁-C₄-alkyl-SO—, C₁-C₄-alkyl-SO₂—, halogen, nitro, cyano or a —OSO₂Y-group,
R⁴, R⁵ each independently of one another denote a —N═N—N(R²)₂-group or a —OSO₂Y-group,
R⁷ denotes hydrogen, methyl, ethyl or a phenyl radical which is substituted by the radicals R⁹, R¹⁰ and is linked directly to the C-atom bearing R⁷,
R⁸ denotes hydrogen, ethyl, CN, NO₂, —CH₂CH₂-halogen or CH₂CH₂OH,
R⁹, R¹⁰ each independently of one another denote hydrogen, hydroxyl, C₁-C₄-alkyl, C₁-C₄-alkoxy, halogen, nitro, cyano, a —N═N—N(R²)₂-group or a —OSO₂Y-group,
Y denotes OH or N(R¹)₂,
R¹ denotes hydrogen, methyl or ethyl,
R² denotes methyl or ethyl.

34. The medicament according to claim 33,
wherein, per total molecule of general formula (1), there are present one or two groups —N═N—N(R²)₂— and one or two —OSO₂Y-groups on any ring-carbon atom of aromatic rings.

35. A medicament for the treatment of a viral disease,
wherein the medicament comprises, together with a pharmaceutically acceptable, at least one antiviral agent comprising at least one diphenyl derivative including its salts, hydrates, solvates, free acids or solvates of these salts or solvates of these free acids or isomers, esters, prodrugs and metabolites,
wherein the diphenyl derivative is a diphenyl derivative represented by one of the following formulae (IV-A1) or (IV-A2): 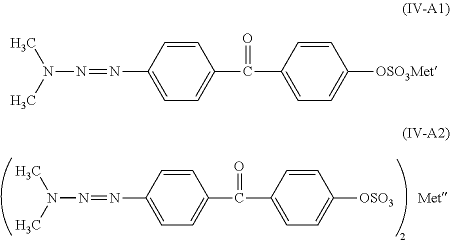
wherein:
Met′ denotes a monovalent metal ion or hydrogen,
Met″ denotes a divalent metal ion.

36. The medicament according to claim 35,
wherein the viral disease is selected among corona infections.

37. The medicament according to claim 35,
wherein the viral disease is COVID-19.

38. The medicament according to claim 35,
wherein the diphenyl derivative is a diphenyl derivative represented by the following formula (IV-A3):

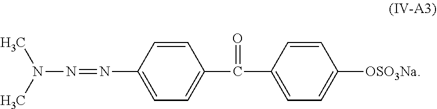

39. The medicament according to claim 35,
wherein the diphenyl derivative is a diphenyl derivative represented by the following formula (IV-A):

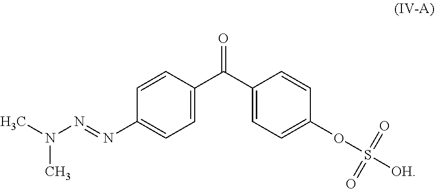 [SMILES: CN(C)/N=N/c1ccc(C(=O)c2ccc(OS(=O)(=O)O)cc2)cc1]

40. A method for the treatment of a patient suffering from a viral disease selected among corona infections,
wherein the method comprises a step of administering to the patient a pharmaceutically effective amount of a medicament as defined in claim 31.

41. The method according to claim 40,
wherein the viral disease is COVID-19.

42. A method for the treatment of a patient suffering from a viral disease, wherein the method comprises a step of administering to the patient a pharmaceutically effective amount of at least one diphenyl derivative including its salts, hydrates, solvates, free acids or solvates of these salts or solvates of these free acids or isomers, esters, prodrugs and metabolites,
wherein the diphenyl derivative is a diphenyl derivative represented by one of the following formulae (IV-A1) or (IV-A2): 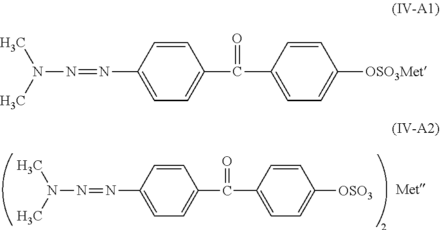
wherein:
Met′ denotes a monovalent metal ion or hydrogen,
Met″ denotes a divalent metal ion.

43. The method according to claim 42,
wherein the viral disease is selected among corona infections.

44. The method according to claim 42,
wherein the viral disease is COVID-19.

45. The method according to claim 42,
wherein the diphenyl derivative is a diphenyl derivative represented by the following formula (IV-A3):

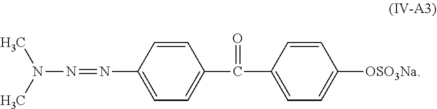

46. The method according to claim 42,
wherein the diphenyl derivative is a diphenyl derivative represented by the following formula (IV-A):

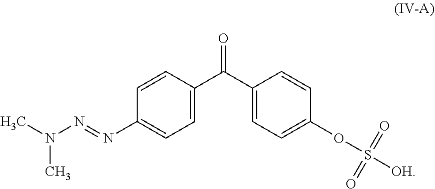 [SMILES: CN(C)/N=N/c1ccc(C(=O)c2ccc(OS(=O)(=O)O)cc2)cc1]

47. A pharmaceutical combination for the treatment of a viral disease, wherein the pharmaceutical combination comprises:
(A) at least one diphenyl derivative including its salts, hydrates, solvates, free acids or solvates of these salts or solvates of these free acids or isomers, esters, prodrugs and metabolites,
wherein the diphenyl derivative is a diphenyl derivative represented by one of the following formulae (IV-A1) or (IV-A2): 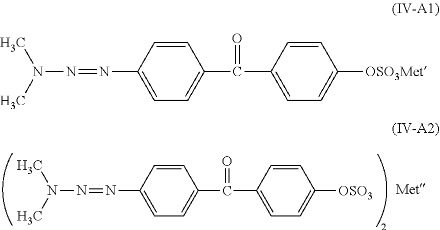
wherein:
Met′ denotes a monovalent metal ion or hydrogen,
Met″ denotes a divalent metal ion;
(B) at least one further active ingredient selected from anti-inflammatory active agents, corticosteroids, blood-thinning active agents, antiviral active agents as well as combinations thereof.
